(19) United States
(12) Patent Application Publication
(10) Pub. No.: US 2013/0204043 A1
(43) Pub. Date: Aug. 8, 2013

(54) METHOD FOR CONTINUOUSLY PRODUCING NITROBENZENE

(71) Applicants: Thomas Knauf, Dormagen (DE); Michael Merkel, Dusseldorf (DE); Andreas Karl Rausch, Kaarst (DE); Peter Lehner, Houston, TX (US); Jürgen Münnig, Kaarst (DE)

(72) Inventors: Thomas Knauf, Dormagen (DE); Michael Merkel, Dusseldorf (DE); Andreas Karl Rausch, Kaarst (DE); Peter Lehner, Houston, TX (US); Jürgen Münnig, Kaarst (DE)

(73) Assignee: Bayer Intellectual Property GmbH Creative Campus Monheim, Monheim (DE)

(21) Appl. No.: 13/812,690

(22) PCT Filed: Jul. 26, 2011
(86) PCT No.: PCT/EP2011/062833

§ 371 Date: Apr. 8, 2013

(30) Foreign Application Priority Data
Jul. 30, 2010 (DE) 102010038678.2

(51) Int. Cl. C07C 201/14

(52) U.S. Cl. CPC C07C 201/14
USPC 564/420; 568/939

(57) ABSTRACT

The present invention relates to a continuous process for the production of nitrobenzene by nitration of benzene with nitric acid or mixtures of nitric acid and sulfuric acid to give a crude nitrobenzene, washing of the crude nitrobenzene by means of at least one of each of an acid, alkaline and neutral washing, there being obtained a pre-purified nitrobenzene which, as well as containing nitrobenzene, at least contains also low boilers, optionally middle boilers as well as high boilers and salts, wherein the pre-purified nitrobenzene is purified further by separating off low boilers in a distillation apparatus by evaporation of the low boilers, and separation of nitrobenzene from the resulting further purified nitrobenzene in a distillation apparatus by partial evaporation of nitrobenzene, wherein pure nitrobenzene is removed from the distillation apparatus in gaseous form and is subsequently condensed, and wherein the non-evaporated portion of the further purified nitrobenzene is fed back into the washing at any desired point.

The present invention relates to a continuous process for the production of nitrobenzene by nitration of benzene with nitric acid or mixtures of nitric acid and sulfuric acid to give a crude nitrobenzene, washing of the crude nitrobenzene by means of at least one of each of an acid, alkaline and neutral washing, there being obtained a pre-purified nitrobenzene which, as well as containing nitrobenzene, at least contains also low boilers, optionally middle boilers as well as high boilers and salts, wherein the pre-purified nitrobenzene is purified further by separating off low boilers in a distillation apparatus by evaporation of the low boilers, and separation of nitrobenzene from the resulting further purified nitrobenzene in a distillation apparatus by partial evaporation of nitrobenzene, wherein pure nitrobenzene is removed from the distillation apparatus in gaseous form and is subsequently condensed, and wherein the non-evaporated portion of the further purified nitrobenzene is fed back into the washing at any desired point.

Nitrobenzene is an important intermediate of the chemical industry which is required in particular for the production of aniline and accordingly for the production of methylenediphenyl diisocyanate (MDI) and the polyurethanes based thereon.

The nitration of benzene with nitric acid to give a crude nitrobenzene has already been the subject of many publications and patent applications. The processes that are conventional today correspond substantially to the concept of the adiabatic nitration of benzene by means of a mixture of sulfuric acid and nitric acid (so-called mixed acid). Such a process was claimed for the first time in U.S. Pat. No. 2,256,999 and is described in current forms, for example, in EP 0 436 443 B1, EP 0 771 783 B1 and U.S. Pat. No. 6,562,247 B2. The processes with an adiabatic reaction procedure are distinguished in particular in that no technical measures are taken to supply heat to the reaction mixture or dissipate heat therefrom.

Isothermal processes for the nitration of benzene with mixed acid are also described, as described, for example, in EP 0 156 199 B1.

Processes for the nitration of benzene which are carried out without the use of sulfuric acid are also known. These are described, for example, in U.S. Pat. No. 2,739,174 or U.S. Pat. No. 3,780,116.

Gas-phase processes for the nitration of benzene with nitric acid or nitrogen oxides are also possible in principle, but the yields achievable thereby are still low (EP 0 078 247 B1, EP 0 552 130 B1).

It is a common feature of all the processes that there is initially obtained as reaction product a crude nitrobenzene which contains as impurities nitric acid and—where nitration has been carried out with mixed acid—sulfuric acid and, as organic impurities, dinitrobenzene as well as nitrated oxidation products of benzene, in particular nitrated phenols (nitrophenols). It also contains organic compounds formed from the compounds that were present as impurities in the benzene used (WO 2008/148608 A1). In addition, the crude nitrobenzene also contains metal salts, which can be present in solution in the acid residues or in the crude nitrobenzene (DE 10 2007 059 513 A1).

Numerous investigations in the past were aimed at improving the quality of the crude nitrobenzene and thus increasing the yield in respect of benzene and nitric acid. Thanks to these developments, current adiabatic liquid-phase processes are so refined that they are all able to produce a crude nitrobenzene that has a low content of by-products, that is to say contains only from 100 ppm to 300 ppm dinitrobenzene and from 1500 ppm to 2500 ppm nitrophenols, it being possible for picric acid to make up from 10% to 50% of the nitrophenols.

The crude nitrobenzene contains as impurities also water, benzene as well as nitrophenols and dinitrobenzene and—where nitration was carried out with mixed acid—sulfuric acid, which are separated off by suitable working-up processes such as, for example, washing and distillation steps. A possible form of this working up is described in EP 1 816 117 B1, where the nitrobenzene is subjected to acid washing, alkaline washing, neutral washing and finally purification by distillation. The purpose of the distillation described in EP 1 816 117 B1 is simply to remove water and benzene from the nitrobenzene and not to evaporate nitrobenzene as completely as possible. The “pure” nitrobenzene of such a process is therefore the bottom product of the distillation and is characterised according to EP 1 816 117 B1 in that it has a conductivity of <50 μS/cm, preferably of <25 μS/cm and particularly preferably of <10 μS/cm (for the method of measuring the conductivity see ibidem, paragraph [0025]). Choosing the conductivity measurement as the sole measure of nitrobenzene purity is disadvantageous because it identifies only water-soluble and dissociable compounds. High molecular weight compounds, such as, for example, nitrated biphenyls (see Example 1) as well as non-dissociable metal compounds such as iron oxides, as well as poorly water-soluble compounds such as calcium sulfate, silicates or metal complexes, are not detected and could accordingly remain unnoticed in the nitrobenzene, because the “pure” nitrobenzene in EP 1 816 117 B1 represents only the bottom product of the distillation. High boiling organic compounds and salts cannot be separated off by such a distillation. Salts in particular can present major problems in the further use of the purified nitrobenzene, for example in hydrogenation to aniline (see below), because they lead to the formation of deposits in apparatuses (e.g. evaporators) or—if they enter the reaction zone—can result in premature deactivation of the catalyst.

When mention is made in the literature relating to nitrobenzene production of distillation of the nitrobenzene, this almost always relates to the separating off by distillation of water and benzene, while the nitrobenzene itself is the bottom product of such a distillation in the processes conventional in the prior art. Some specifications even expressly advise against evaporation of the nitrobenzene itself for safety reasons. For example, U.S. Pat. No. 4,021,498 describes a particular process for the adiabatic nitration of benzene with the aim of minimising the content of dinitrated compounds in the product (see column 2, lines 18 to 29). U.S. Pat. No. 4,021,498 expressly mentions the risks that accompany evaporation of the nitrobenzene itself (column 2, lines 7 to 17), and its teaching is accordingly directed away from purification of the nitrobenzene by evaporation and recondensation.

Only a small number of publications relating to the production and purification of nitrobenzene actually teach evaporation of the nitrobenzene itself. U.S. Pat. No. 1,793,304, for example, describes a process for the purification of (inter alia) nitrobenzene in which contaminated nitrobenzene is purified first by filtration through so-called fuller's earth filters and then by heat treatment with aluminium oxide or other basic oxides at from 80° C. to 100° C. (p. 1, 1. 63 to 71). The treatment with aluminium oxide is preferably followed by distillation under a partial vacuum at bottom temperatures of from 140° C. to 160° C. (p. 1, 1. 74 to 79). Further details of the implementation of the distillation are not given in the publication; however, on the basis of the boiling point of nitrobenzene at normal pressure (211° C.), it can be assumed that nitrobenzene is actually evaporated in the mentioned temperature range and under a “partial vacuum”. The specification provides no information regarding the recovery of the energy used in the distillation.

U.S. Pat. No. 2,431,585 describes a gas-phase nitration of benzene with subsequent distillation of the nitrobenzene at the head of the distillation column 14 (see figure). A partial discharge of the distillation bottoms is not provided.

CH 186266 discloses a process for the purification of commercial nitrobenzene in which, before the nitrobenzene is distilled off, impurities such as dinitrobenzene are converted by chemical reaction with basic compounds and organic substances (see dependent claim 1, Examples 2 to 6) into different compounds which can readily be separated off The indicated distillation conditions are 122° C. and 66 mbar (see Example 1). At the level of such distillation temperatures, the heat of condensation cannot expediently be recovered, for example in the form of usable steam.

RU 2 167 145 C1 describes the separation of high boiling compounds—in particular sulfur-containing organic compounds—from nitrobenzene by rectification of the nitrobenzene, nitrobenzene being removed at the head of the rectification column. However, this specification does not discuss the particular problems of salts in the crude nitrobenzene, which can lead to packings and heat exchangers becoming blocked. The distillation conditions chosen in RU 2167145 C1 are also disadvantageous because only low pressures (from 20 mm Hg to 80 mm Hg, corresponding to from 27 mbar to 107 mbar; see claim 1) can be used, which leads to condensation temperatures of only from 99° C. to 134° C., as the person skilled in the art can readily determine by calculation (for example by calculation of the saturation vapour pressure curve with the aid of the Antoine equation, see in this connection I. Brown, Australian Journal of Scientific Research, Series B: Biological Sciences (1952), 5A, 530-540). At this level of condensation temperatures, the heat of condensation cannot be recovered expediently, for example in the form of usable steam.

CN 100999472 A describes the working up of the bottom residue of nitrobenzene distillation in order to obtain m-dinitrobenzene, without going into greater detail regarding the conditions of the distillation of the nitrobenzene.

The purified nitrobenzene (pure nitrobenzene) is used primarily in the production of aniline, which today takes place predominantly by catalytic hydrogenation of the nitrobenzene in the gas phase with hydrogen. For conversion into the gas phase, nitrobenzene can either be evaporated (EP 0 696 574 B1, paragraph [0024]) or atomised into a hot gas stream, preferably into a hydrogen stream (DE-OS-1 809 711, DE 10 2006 035 203 A1, paragraph [0053]). Evaporation into the fresh hydrogen stream is considered to be advantageous because markedly fewer deposits are said to form in the reactor and in the piping (EP 0 696 574 B1, paragraph [0024]). However, evaporation into the fresh hydrogen stream does not avoid the formation of deposits in the reactor completely since metal compounds, salts and high boilers present in the nitrobenzene will continue to occur there. The occurrence of solids (such as high boiling organic compounds and salts) in the reactor is disadvantageous because the cleaning intervals of the reactor are shortened as a result and catalyst can be deactivated prematurely. Although problems with deposits in the reactor can be minimised if the nitrobenzene is evaporated in a conventional evaporator, they shift to the evaporator stage: Necessary frequent cleaning of the evaporator repeatedly leads to interruptions in the continuous operation of the aniline plant. In addition, depending on the quality of the nitrobenzene used, the above-mentioned safety problems occasionally occur. There is therefore a need for a production method for nitrobenzene which provides nitrobenzene with the necessary purity from the outset without further purification steps being required in process steps of the aniline production.

The hydrogenation of nitrobenzene to aniline can be carried out on fixed catalyst beds in tubular reactors (DE-AS-2 201 528) or staged reactors (EP 0 696 574 B1, paragraph [0021]) or in fluidised bed reactors (DE-AS-1 114 820). Catalysts for the gas-phase hydrogenation of nitroaromatic compounds are described in many publications. The hydrogenation-active elements include Pd, Pt, Ru, Fe, Co, Ni, Cu, Mn, Re, Cr, Mo, V, Pb, Ti, Sn, Dy, Zn, Cd, Ba, Cu, Ag, Au. It is likewise possible to use these elements as hydrogenation catalyst in the form of their compounds, for example as oxides, sulfides or selenides and also in the form of a Raney alloy as well as on supports, such as Al₂O₃, Fe₂O₃/Al₂O₃, SiO₂, silicates, carbon, TiO₂, Cr₂O₃.

The reactors can be operated isothermally with the use of cooling (DE-AS-2 201 528) or also adiabatically (EP 0 696 574 B1). Combinations of isothermal and adiabatic reaction sections are also possible (GB 1 452 466).

All publications in which the evaporation of nitrobenzene is disclosed, whether it be within the context of a nitrobenzene synthesis in a distillation apparatus or within the context of the evaporation of the starting material nitrobenzene in a gas-phase aniline synthesis, have the common feature that it is desired to evaporate the nitrobenzene as completely as possible. This has the result on the one hand that safety problems can occur during the evaporation (see above) and on the other hand that deposits of high boiling organic secondary components and salts are formed in evaporators and distillation apparatuses, which interfere with the operation and can lead to stoppages in production.

Accordingly, it was an object of the present invention to configure the working up of crude nitrobenzene so that it can be carried out in a manner that is as trouble-free (no plant failure due, for example, to the necessity of removing salt deposits in a distillation column), safe (no problems as a result of the accumulation of potentially explosive compounds in the bottom of a distillation column) and energy-efficient (optimum utilisation of heat obtained in the process) as possible.

It was a further object of the present invention to provide a pure nitrobenzene in which high boiling organic compounds and salts have either been removed completely or have at least been depleted to such an extent that this pure nitrobenzene can be employed in further uses without disadvantages.

In particular, it was also an object of the present invention to provide a pure nitrobenzene with which an aniline process can be operated with long reactor service lives (=long cleaning intervals). In order to provide such a nitrobenzene, the quality of the nitrobenzene cannot be assessed—as described in the prior art—solely on the basis of its conductivity. For example, it has been found that a nitrobenzene whose conductivity was less than 10 μS/cm and which accordingly would be described as pure within the scope of EP 1 816 117 B1 can still contain up to 200 ppb (0.2 ppm) sodium. The use of such a nitrobenzene in a gas-phase aniline process is disadvantageous because contamination of the nitrobenzene heater and evaporator or, in the case of atomisation of the nitrobenzene, contamination of the catalyst surface can occur, which can result in a reduction in the selectivity and/or lifetime of the catalyst (see Examples 4 and 5).

It has been found that the object can be achieved by a process for the production of nitrobenzene by
a) nitration of benzene with nitric acid or mixtures of nitric acid and sulfuric acid and subsequent separation of excess acid used in the nitration, wherein crude nitrobenzene is obtained,
b) washing of the crude nitrobenzene from step a) by means of at least one of each of an acid, alkaline and neutral washing, preferably in that sequence, wherein there is obtained after separation of the wash fluid used in the last washing a pre-purified nitrobenzene which, in addition to containing nitrobenzene, also contains at least low boilers, optionally middle boilers as well as high boilers and salts,
c) separation of low boilers from the pre-purified nitrobenzene from step b) in a distillation apparatus by evaporation of the low boilers, wherein the bottom product nitrobenzene is depleted of low boilers and thus further purified,
d) partial separation of nitrobenzene from the further purified nitrobenzene from step c) in a distillation apparatus by partial evaporation of nitrobenzene, preferably by evaporation of from >80% by mass to ≦99.9% by mass, preferably from >90% by mass to ≦95% by mass, of the further purified nitrobenzene, based on the total mass of the further purified nitrobenzene, wherein pure nitrobenzene is removed from the distillation apparatus in gaseous form and is subsequently condensed, and non-evaporated nitrobenzene, optionally middle boilers as well as high boilers and salts are discharged partially to completely, preferably completely, from the bottom of the distillation apparatus, and
e) return of the non-evaporated portion of the further purified nitrobenzene from step d) into any desired point of step b), preferably into the acid washing.

Excess acid used in the nitration refers to any type of unreacted acid, that is to say

optionally nitric acid when the nitration is carried out only with nitric acid

or

sulfuric acid or optionally nitric acid and sulfuric acid when the nitration is carried out with mixtures of nitric acid and sulfuric acid.

Within the context of this invention, low boilers denotes any compounds and azeotropically boiling mixtures of compounds whose boiling points are below the boiling point of nitrobenzene under the chosen distillation conditions. The main constituent of the low boilers is incompletely reacted benzene. Further typical low boilers are n-hexane, cyclohexane, n-heptane, methylcyclohexane, bicycloheptane and the isomeric dimethylpentanes.

Within the context of this invention, middle boilers denotes any compounds and azeotropically boiling mixtures of compounds whose boiling points are above the boiling point of nitrobenzene under the chosen reaction conditions but below 350° C. at normal pressure. Typical middle boilers are the isomeric dinitrobenzenes.

Within the context of this invention, high boilers denotes any compounds and azeotropically boiling mixtures of compounds whose boiling points at normal pressure are above 350° C. The boiling points of such compounds are so high that contamination and deactivation of the catalyst cannot be ruled out in a catalysed gas-phase hydrogenation to aniline, which is the preferred use of the nitrobenzene produced according to the invention. Typical high boilers have more than one aromatic ring and include, for example, nitrated biphenyls as well as nitrated hydroxy-biphenyls.

Typical salts which are separated from the nitrobenzene by the process according to the invention are the sodium and calcium salts of nitrite, nitrate, sulfate and oxalate as well as silicon oxide and iron oxide.

By means of the procedure according to the invention, extraordinarily high degrees of purity of the nitrobenzene are achieved. Nitrobenzene produced by the process according to the invention has a degree of purity in relation to organic impurities (substantially the above-mentioned middle boilers) of >99.9500% (corresponding to <500 ppm impurities), preferably of >99.9900% (corresponding to <100 ppm impurities), particularly preferably of >99.9990% (corresponding to <10 ppm impurities) (preferably determined by means of gas chromatography) and contains not more than 0.1 ppm, preferably not more than 0.05 ppm, particularly preferably not more than 0.01 ppm, inorganic impurities (salts, determined as cations, by atom absorption spectrometry (inductive coupled plasma, ICP)). (Unless indicated otherwise, all contents indicated in % and ppm are always based on the mass).

A highly pure nitrobenzene produced by the process according to the invention is suitable in particular for use in hydrogenation to aniline.

Step a) of the process according to the invention can in principle be carried out by any methods known in the prior art. Preference is given to the reaction of benzene with a mixture of nitric acid and sulfuric acid in an adiabatic procedure, as is described in DE 10 2008 048713 A1, paragraph [0024], which is accordingly considered to be part of the present disclosure.

Step b) of the process according to the invention is known in principle in the prior art. In this three-stage washing, the crude nitrobenzene obtained in step a) is washed with suitable wash fluids (i.e. wash waters having a pH corresponding to the stage in question) in three washing steps, it being possible in principle for the individual washing steps to be carried out in any desired sequence. However, preference is given to the sequence (1) acid washing—(2) alkaline washing—(3) neutral washing. Particularly preferably, step b) is carried out according to the procedure described in Paragraphs [0008] to [0012] of EP 1 816 117 B1, which is accordingly considered to be part of the present disclosure. Particularly preferably, there is used in the neutral washing in step b) an electrophoresis as described in EP 1 816 117 B1, paragraph [0013], which is accordingly likewise considered to be part of the present disclosure.

In step c) of the process according to the invention there is used as the distillation apparatus preferably a rectification column, that is to say an apparatus in which at least one theoretical plate is produced and in which a liquid reflux is fed into the top of the column. The reflux ratio, that is to say the ratio of reflux to withdrawn condensate, is preferably from 0.01 to 0.5, and there is preferably used as reflux one (or more) low boiler(s), particularly preferably benzene. The benzene preferably used as reflux can be fresh benzene or, particularly preferably, benzene obtained from the top product of the rectification column after separation of the water (e.g. by static phase separation). Furthermore, the benzene preferably used as reflux does not necessarily have to be pure benzene. In fact, it can also be a mixture which contains, in addition to benzene, from 0.1% by mass to 10% by mass nitrobenzene and/or from 0.1% by mass to 50% by mass aliphatic hydrocarbons, in each case based on the total mass of the reflux. In step c) of the process according to the invention, nitrobenzene is the bottom product, that is to say only nitrobenzene from the bottom of step c) is distilled off in step d). (Negligible amounts of nitrobenzene may be carried along with the low boilers in step c) and, if they do not enter the bottom again with the reflux, can be discharged from the system.) In step d) of the present invention, high boilers and salts are separated off by subjecting the further purified nitrobenzene obtained in step c) to a distillation process in which pure nitrobenzene is removed from the distillation apparatus in gaseous form and is subsequently condensed, that is to say the nitrobenzene itself is distilled off in this step. Preferably, the pure nitrobenzene is removed from the distillation column at the head. However, removal as a side stream is also conceivable. The bottom product formed in the distillation apparatus accounts for from >0.1% by mass to 20% by mass, preferably from >5 to 10% by mass, of the further purified nitrobenzene that is introduced into the distillation apparatus in step d). The partial separation of nitrobenzene from the further purified nitrobenzene in a distillation apparatus by partial evaporation of nitrobenzene according to the invention therefore means that in step d) from >80% by mass to ≦99.9% by mass, preferably from >90% by mass to ≦95% by mass, of the further purified nitrobenzene, based on the total mass of the further purified nitrobenzene, are evaporated.

In step e), the distillation bottoms formed in step d) (that is to say the non-evaporated portion of the further purified nitrobenzene) are fed to the washing (step b)) at any desired point. This procedure according to the invention, in which the bottoms of the distillation column in step d) are not evaporated completely, on the one hand increases the safety of the process. On the other hand, problems with the deposition of solids in apparatuses are avoided because a still liquid bottom product is discharged and fed back into the washing (step b)). Salts are washed out there, and nitrobenzene discharged with the bottoms is transferred, apart from negligible residual contents, into the organic phase of the washing stage in question, that is to say it is fed back into the pre-purified nitrobenzene. Losses of yield are thereby minimised.

The pressure in the distillation step d) is preferably so chosen that a condensation temperature of the pure nitrobenzene is obtained at which the heat of condensation can be used economically for the generation of steam. Accordingly, the invention also relates in particular to a process in which absolute pressures in the range from 150 mbar to 1000 mbar, preferably from 200 mbar to 600 mbar, particularly preferably from 400 mbar to 500 mbar, are maintained in step d) and in which the heat that is to be dissipated in the condensation of the pure nitrobenzene in step d) is used to generate steam. The pressure prevailing in step d) is preferably measured at the condenser in which the pure nitrobenzene leaving the distillation apparatus in gaseous form is condensed. All condensers conventional according to the prior art can be used for this purpose, preference being given to tubular heat exchangers or plate heat exchangers.

With such pressures in step d), condensation temperatures of the pure nitrobenzene of from 140° C. to 210° C., preferably from 150° C. to 190° C., particularly preferably from 175° C. to 185° C., are established. In the process according to the invention, a temperature of 210° C. is preferably not exceeded at any point in the condensation of the pure nitrobenzene in step d), particularly preferably throughout step d), most particularly preferably throughout steps a) to d). Only if the pure nitrobenzene is condensed at temperatures not exceeding 210° C. can it be obtained free of or at least very low in high boiling compounds, in particular organic high boiling compounds (“high boilers”). It has in fact been found that high boilers form from nitrobenzene at temperatures >210° C. (see Example 2). Therefore, it is surprisingly only possible to provide salt-free nitrobenzene which at the same time is also free of or at least very low in high boilers, while at the same time maximising the yield (based on benzene), when the maximum temperature at which the nitrobenzene is condensed (which is generally the liquid phase temperature in the condenser) does not exceed 210° C. However, a high condensation temperature is in principle desirable because it is only possible to use the heat of condensation economically for steam generation if the condensation temperature is sufficiently high. Therefore, the condensation temperature that is actually to be established is generally a compromise and preferably ensures an absolute pressure of the steam that is obtained of at least 4 bar, which corresponds to a condensation temperature of 150° C.

Because the preferred use of the pure nitrobenzene according to the invention is hydrogenation to aniline, particular attention must be paid when separating off high boilers in step d) to compounds that are not gaseous under the conditions of a gas-phase aniline process. The aniline production preferably takes place according to the process of DE 10 2006 035 203 A1. Particularly preferably, the ranges of temperature, pressure, water content in the starting gas stream and hydrogen excess mentioned in paragraph [0018] therein are maintained. Nitrobenzene is preferably converted into the gas phase by means of atomisation as described in paragraph [0053]. The mentioned paragraphs are accordingly considered to be part of the present disclosure. High boiling organic compounds which are not gaseous under the conditions of such a gas-phase aniline process, and salts present in the pre-purified nitrobenzene, can be separated off in step d) of the nitrobenzene working up upstream of the aniline production, preferably in a distillation apparatus without separation-active internals. In the case of distillation without separation-active internals, the term evaporation is also used. However, the distillation apparatus preferably has demisters or droplet separators.

Accordingly, particular preference is given to a process in which the distillation apparatus in step c) is a rectification column which is operated with partial reflux of low boilers, preferably benzene, particularly preferably benzene containing nitrobenzene and/or aliphatic hydrocarbons, and in step d) is an apparatus without separation-active internals. It is also possible to dispense with a reflux (which in this case would be a portion of the condensed pure nitrobenzene) in step d). Accordingly, the invention relates in this embodiment to a process in which the distillation apparatus in step c) is a rectification column which is operated with partial reflux of low boilers, preferably benzene, particularly preferably benzene containing nitrobenzene and/or aliphatic hydrocarbons, and in step d) is an apparatus without separation-active internals which is operated without reflux of condensed pure nitrobenzene. By means of this particularly preferred form of the two-stage (steps c) and d)) distillation according to the invention it is ensured on the one hand that the low boilers are separated efficiently from the nitrobenzene (rectification in step c)) and on the other hand that the separation of high boilers and in particular salts in step d) takes place without the risk of packings in the distillation column becoming blocked. Any middle boilers not completely separated off in step d) are present in the pure nitrobenzene only in non-critical amounts of <500 ppm, preferably <100 ppm, particularly preferably <10 ppm, based on the mass of the pure nitrobenzene.

Because the formation of high boiling organic compounds (such as the mentioned nitrated biphenyls) can also take place at temperatures lower than 210° C. if the nitrobenzene is exposed to such temperatures for too long, it is advantageous for the apparatuses used to have a short residence time. For example, self-circulation evaporators, e.g. so-called Robert evaporators, can be used as evaporators for the distillation columns in steps c) and d) (see Reinhard Billet: Verdampfung and ihre technischen Anwendungen, Weinheim, 1981, p. 119 ff). The residence time of the nitrobenzene in such evaporators is preferably from 0.1 minute to 120 minutes, particularly preferably from 0.1 minute to 20 minutes.

The separation of low and high boiling compounds and salts from the nitrobenzene can be combined in a dividing wall column. (The general principle of operation of dividing wall columns is described, for example, in G. Kaibel, “Distillation Columns with Vertical Partitions”, Chem. Eng. Technol. 1987, 10, 92-98 and G. Kaibel, “Industrieller Einsatz von Trennwandkolonnen and thermisch gekoppelten Destillationskolonnen”, Chemie Ingenieur Technik 2003, 75, 1165-1166.) Accordingly, the invention relates in this embodiment in particular to a process in which the same distillation apparatus is used in steps c) and d) and the distillation apparatus is a dividing wall column.

### EXAMPLES

Example 1 (According to the Invention)

### Separation of High Boilers by Distillation of the Bitrobenzene—Step d)

277.6 g of a nitrobenzene containing no detectable amounts of low boilers and 55 ppm per unit area of high boilers (determined by gas chromatography, GC) were distilled at 180° C. and 400 mbar (absolute) in a distillation apparatus consisting of a glass flask and a distillation bridge (step d) of the process according to the invention) so that 271.4 g of distillate and 6.2 g as bottom product were obtained. About 3700 ppm per unit area of high boilers were found in the distillation bottoms by means of GC. In the group of the high boilers it was possible to identify by means of GC-MS (mass spectrometry) 2-nitrobiphenyl and 2,2-dinitrobiphenyl. No high boilers could be found in the distillate (=pure nitrobenzene), which shows that a nitrobenzene that is free of high boilers can be obtained at low distillation temperatures (<210° C.) by means of the process according to the invention.

### Example 2 (Comparison Example)

### Formation of High Boilers by Tempering

In a stainless steel autoclave, nitrobenzene of >99.9900% purity (GC) and having a salt content of <0.1 ppm (which can accordingly be referred to within the context of the present application as being free of low boilers, high boilers and salts) was exposed to an elevated temperature for a period of 2 hours. After cooling, the nitrobenzene was analysed by gas chromatography for the content of high boiling impurities. The results are given in Table 1 and show that high boiling compounds were formed solely due to the thermal load and that nitrobenzene thus cannot be exposed to arbitrarily high temperatures if it is to be obtained in a form free of high boilers.

TABLE 1

| | | | |
|---|---|---|---|
| Tempering temperature [° C.] | 240 | 260 | 280 |
| Tempering time [h] | 2 | 2 | 2 |
| Amount of high boilers | 193 | 303 | 589 |
| [ppm by unit area] | | | |

### Examples 3 to 5

### Relating to the Use of Nitrobenzene of Different Salt Contents in Hydrogenation to Aniline

The test system used for the exemplary reactions is a 500 mm long reaction tube of stainless steel. A circulating gas stream, which is heated to 250° C. by means of a heat exchanger, is passed through the reactor. Nitrobenzene is fed to a nozzle by means of metering pumps and finely atomised in the circulating gas stream, where it then evaporates. Hydrogen is pre-heated in a heat exchanger and metered into the circulating gas upstream of the nozzle. The hydrogen supply is regulated by a mass flow regulator. The load of the catalyst in the reaction tube was adjusted to a value of 1.0 g_{nitroaromatic compound}/(ml_{catalyst}·h) in all the exemplary tests, and the hydrogen:nitrobenzene ratio in the reactor was set at about 80:1.

A 400 mm high packed bed of the catalyst was placed on a screen inside the reaction tube. After leaving the reactor, the reaction product is cooled with water. The non-volatile constituents are thus condensed out and separated from the gaseous components in a downstream separator. The liquid constituents are guided from the separator into the product collection vessel and collected therein (glass container). Upstream of the collection vessel there is a sample removal point at which samples of the product can be taken at regular intervals. These are analysed by gas chromatography. The service life of the catalyst corresponds to the time from the beginning of the reaction until complete conversion of the nitrobenzene is no longer achieved and >0.1% nitrobenzene is detected in the product at the sample removal point by means of gas chromatography.

In each of Examples 3 to 5 there was used a nitrobenzene which contains about 250 ppm organic impurities and accordingly meets the requirements according to the invention as regards the content of organic impurities (<500 ppm). However, the nitrobenzene qualities in Examples 3 to 5 differ in terms of their salt content (exemplified by the content of sodium ions).

The examples were carried out with the catalyst system 9 g/lₛᵤₚₚₒᵣₜ Pd, 9 g/lₛᵤₚₚₒᵣₜ V, 3 g/lₛᵤₚₚₒᵣₜ Pb on α-aluminium oxide (see EP 0 011 090 A1). Differently aged and pre-treated catalysts of this catalyst system were used; the reaction tests were each terminated when the service life of the catalyst was reached.

### Example 3 (According to the Invention)

### Use of Nitrobenzene having a Content of Sodium Ions of <0.1 ppm

Fresh catalyst was placed in the reaction tube and flushing was carried out first with nitrogen and then with hydrogen. The catalyst was then subjected to 1000 l/h of hydrogen for a period of 48 hours at 240° C. Evaporated nitrobenzene was then guided onto the catalyst. The nitrobenzene load was increased slowly to the desired value of 1 g_{nitroaromatic compound}/(ml_{catalyst}·h) so that the temperature in the reactor did not rise above 450° C., and the hydrogen addition was so adjusted that the molar ratio hydrogen:nitrobenzene was 80:1. As soon as the conversion of nitrobenzene was no longer complete (more than 0.1% nitrobenzene in the reaction product), the feed of starting material was terminated and the reactor was rendered inert with nitrogen. Carbon deposits were then burnt off at 270° C. in the air stream until less than 0.2 vol. % CO₂ could be detected in the exhaust gas. This cycle of reaction and regeneration of the catalyst was carried out a total of 3 times (Examples 3a to 3c). The service lives were 983 hours, 964 hours and 968 hours, respectively.

### Example 4 (Comparison Example)

### Use of Nitrobenzene having a Content of Sodium Ions of 0.2 ppm

Fresh catalyst was placed in the reaction tube and flushing was carried out first with nitrogen and then with hydrogen. The catalyst was then subjected to 1000 l/h of hydrogen for a period of 48 hours at 240° C. Evaporated nitrobenzene was then guided onto the catalyst. The nitrobenzene load was increased slowly to the desired value of 1.0 g_{nitroaromatic compound}/(ml_{catalyst}·h) so that the temperature in the reactor did not rise above 450° C., and the hydrogen addition was so adjusted that the molar ratio hydrogen:nitrobenzene was 80:1. As soon as the conversion of nitrobenzene was no longer complete (more than 0.1% nitrobenzene in the reaction product), the feed of starting material was terminated and the reactor was rendered inert with nitrogen. Carbon deposits were then burnt off at 270° C. in the air stream until less than 0.2 vol. % CO₂ could be detected in the exhaust gas. This cycle of reaction and regeneration of the catalyst was carried out a total of 3 times (Examples 4a to 4c). The service lives were 953 hours, 848 hours and 736 hours, respectively, and were accordingly markedly poorer—in particular in the 2nd and 3rd cycles—than when a nitrobenzene produced according to the invention was used.

### Example 5 (Comparison Example)

### Use of Nitrobenzene having a Content of Sodium Ions of 0.4 ppm

Fresh catalyst was placed in the reaction tube and flushing was carried out first with nitrogen and then with hydrogen. The catalyst was then subjected to 1000 l/h of hydrogen for a period of 48 hours at 240° C. Evaporated nitrobenzene was then guided onto the catalyst. The nitrobenzene load was increased slowly to the desired value of 1.0 g_{nitroaromatic compound}/(ml_{catalyst}·h) so that the temperature in the reactor did not rise above 450° C., and the hydrogen addition was so adjusted that the molar ratio hydrogen:nitrobenzene was 80:1. As soon as the conversion of nitrobenzene was no longer complete (more than 0.1% nitrobenzene in the reaction product), the feed of starting material was terminated and the reactor was rendered inert with nitrogen. Carbon deposits were then burnt off at 270° C. in the air stream until less than 0.2 vol. % CO₂ could be detected in the exhaust gas. This cycle of reaction and regeneration of the catalyst was carried out a total of 3 times (Examples 5a to 5c). The service lives were 946 hours, 768 hours and 605 hours, respectively, and were accordingly even poorer than in Example 4.

The following table compares the results of Examples 2 to 4 again:

TABLE 2

| According to | | | | | |
|---|---|---|---|---|---|
| the invention | | Comparison | | | |
| Exam- | Service life | Exam- | Service life | Exam- | Service life |
| ple | [h] | ple | [h] | ple | [h] |
| 3a | 983 | 4a | 953 | 5a | 946 |
| 3b | 964 | 4b | 848 | 5b | 768 |
| 3c | 968 | 4c | 736 | 5c | 605 |

### Example 6 (According to the Invention)

### Energy Recovery in the Condensation

By means of an Aspen simulation, the mass and energy balance for the additional nitrobenzene purification step d) by means of evaporation and condensation was calculated. 50 t/h of nitrobenzene having a temperature of 183° C. are fed to a continuously operated evaporator. At an absolute system pressure of 500 mbar, 90% of the feed amount (i.e. 90% by mass of the further purified nitrobenzene) are evaporated using 20 bar steam. The remaining 10% are discharged from the evaporator and fed back into the acid washing. The energy required for the evaporation is 4.7 MW or 8.6 t/h 20 bar steam.

The vapours are condensed in a downstream heat exchanger which is operated at 490 mbar absolute on the process side. For cooling, condensate having a supply temperature of 100° C. is employed, which is used to generate 6 bar steam. In the condensation step, an energy of 4.7 MW is released. Because the condensate must still be pre-heated from 100° C. to the evaporation temperature of 159° C., 7.2 t/h of 6 bar steam are generated in this apparatus.

By using the heat energy obtained in the condenser, the energy required for the evaporation of the nitrobenzene can be recovered completely. The pressure on the process side is so chosen that the MNB can be evaporated with the higher of the available pressure stages of the steam and steam of the lower of the available pressure stages can be generated in the condenser.

1-7. (canceled)

8. A process for producing nitrobenzene comprising
a) nitrating benzene with nitric acid or a mixture of nitric acid and sulfuric acid to obtain crude nitrobenzene and subsequently separating excess acid from said crude nitrobenzene;
b) washing said crude nitrobenzene from step a) with at least one each of an acid, alkaline, and neutral washing to obtain a mixture comprising pre-purified nitrobenzene and low boilers after the final washing;
c) separating the low boilers from the pre-purified nitrobenzene from step b) in a distillation apparatus by evaporation of the low boilers to obtain a bottom product of further purified nitrobenzene depleted of low boilers;
d) partially separating nitrobenzene from the further purified nitrobenzene from step c) in a distillation apparatus by partial evaporation of nitrobenzene, wherein pure nitrobenzene is removed from the distillation apparatus in gaseous form and is subsequently condensed; and
e) returning the non-evaporated portion of the further purified nitrobenzene from step d) into any stage of step b).

9. The process of claim 8, wherein an absolute pressures in the range from 150 mbar to 1000 mbar is maintained in step d) and wherein heat dissipated in the condensation of the pure nitrobenzene in step d) is used to generate steam.

10. The process of claim 8, wherein the distillation apparatus in step c) is a rectification column operated with partial reflux of low boilers and in step d) is an apparatus without separation-active internals.

11. The process of claim 10, wherein the distillation apparatus in step d) is operated without reflux of condensed pure nitrobenzene.

12. The process of claim 8, wherein the same distillation apparatus is used in steps c) and d) and the distillation apparatus is a dividing wall column.

13. The process of claim 8, wherein an electrophoresis is used in the neutral washing in step b).

14. A process comprising hydrogenating the nitrobenzene obtained in claim 8 to obtain aniline.
